(19) United States
(12) Patent Application Publication
(10) Pub. No.: US 2023/0355293 A1
(43) Pub. Date: Nov. 9, 2023

(54) TREATMENT PLANNING FOR ELECTRICAL-ENERGY BASED THERAPIES BASED ON CELL CHARACTERISTICS

(71) Applicant: Virginia Tech Intellectual Properties, Inc., Blacksburg, VA (US)

(72) Inventors: Rafael V. Davalos, Blacksburg, VA (US); Christopher B. Arena, Blacksburg, VA (US)

(21) Appl. No.: 18/130,330

(22) Filed: Apr. 3, 2023

Related U.S. Application Data
(63) Continuation of application No. 14/686,380, filed on Apr. 14, 2015.
(60) Provisional application No. 61/979,205, filed on Apr. 14, 2014.

(51) Int. Cl. A61B 18/12

(52) U.S. Cl. CPC A61B 18/1206; A61B 2018/00666

(57) ABSTRACT

A method for treating a target tissue in a patient in need thereof is provided. The method includes the steps of identifying one or more characteristics of one or more cells of a target tissue; calculating a threshold electric field for inducing IRE in the target tissue based on the one or more characteristics; constructing a treatment protocol of one or more pulse parameters, wherein the treatment protocol is capable of inducing IRE in the target tissue; and delivering the treatment protocol to the target tissue. Systems for treatment planning for medical therapies involving administering electrical treatment energy are also provided.

### CROSS REFERENCE TO RELATED APPLICATIONS

The present application relies on the disclosure of and claims priority to and the benefit of the filing date of U.S. Provisional Application No. 61/979,205 filed Apr. 14, 2014, the disclosure of which is hereby incorporated by reference herein in its entirety.

### BACKGROUND OF THE INVENTION

### Field of the Invention

The present invention is related to medical therapies involving the administering of electrical treatment energy. More particularly, embodiments of the present invention provide systems and methods for determining a threshold electric field for inducing cell death and one or more parameters of a treatment protocol for delivering one or more electrical pulses to a target tissue based on one or more cell characteristics (such as cell size) that influence the transmembrane potential of the cells of the target tissue during treatment.

### Description of Related Art

Irreversible electroporation (IRE) is a non-thermal tumor ablation therapy that utilizes pulsed electric fields to create defects in the plasma membrane leading to cell death. IRE offers benefits over other cancer therapies in that it can be performed near large blood vessels, nerves, and ducts without damage to these structures, sparing the extracellular matrix (Onik, G. and B. Rubinsky, eds. Irreversible Electroporation: First Patient Experience Focal Therapy of Prostate Cancer. Irreversible Electroporation, ed. B. Rubinsky 2010, Springer Berlin Heidelberg. 235-247 (“Onik and Rubinsky, 2010”) and Davalos, R. and B. Rubinsky, Tissue ablation with irreversible electroporation, U.S. Pat. Nos. 8,282,631 and 8,048,067). Thermal therapies typically cannot be performed near large blood vessels or other critical structures as blood flow carries heat away from the region of interest (heat sink effect), not allowing high critical temperatures to be reached in order to kill diseased tissue. In addition, IRE allows for submillimeter resolution between treated and un-treated tissue as well as visualization using real-time imaging, allowing the surgeon immediate confirmation of treatment (Rubinsky, B.,G. Onik, and P. Mikus, Irreversible electroporation: a new ablation modality--clinical implications. Technol Cancer Res Treat, 2007, 6(1): p. 37-48; and Zhang, Y., et al., MR imaging to assess immediate response to irreversible electroporation for targeted ablation of liver tissues: preclinical feasibility studies in a rodent model. Radiology, 2010. 256(2): p. 424-32). Characterizing the lesions is necessary for acceptance of IRE as a primary cancer therapy in the clinical setting.

While many parameters are taken into account for a successful IRE treatment, the electric field distribution of the tissue remains the key component that needs to be assessed. There is minimal data on whether different cancer cell lines havedifferent electric field thresholds for cell death (Bower, M., et al., Irreversible electroporation of the pancreas: definitive local therapy without systemic effects. Journal of surgical oncology, 2011. 104(1): p. 22-28; Charpentier, K.P., et al., Irreversible electroporation of the pancreas in swine: a pilot study. HPB : the official journal of the International Hepato Pancreato Biliary Association, 2010. 12(5): p. 348-351; and Martin,n.R.C.G., et al., Irreversible electroporation therapy in the management of locally advanced pancreatic adenocarcinoma. Journal of the American College of Surgeons, 2012. 215(3): p. 361-369). Currently, electric field thresholds are available for in vivo liver tissue, brain tissue, and orthotopic mammary tumors in mice (Edd, J.F., et al., Invivo results of a new focal tissue ablation technique: irreversible electroporation. IEEE transactions on bio-medical engineering, 2006. 53(7): p. 14091415.;. Miklavcic, D., et al., A validated model of in vivo electric field distribution in tissues for electrochemotherapy and for DNA electrotransfer for gene therapy. BBA – General Subjects, 2000. 1523(1): p. 73-83; Sano, M., et al., Towards the creation of decellularized organ constructs using irreversible electroporation and active mechanical perfusion. BioMedical Engineering OnLine, 2010. 9(1): p. 83.; Garcia, P.A., et al., Intracranial nonthermal irreversible electroporation: in vivo analysis. The Journal of membrane biology, 2010. 236(1): p. 127-136.; Neal li, R.E., et al., Treatment of breast cancer through the application of irreversible electroporation using a novel minimally invasive single needle electrode. Breast Cancer Research and Treatment, 2010. 123(1): p. 295-301). While clinical trials for prostate, pancreatic, kidney, liver, and lung cancer are in phase I clinical trials, more information about the electric field thresholds required to kill cancer cells are necessary as well as to improve treatment planning algorithms (Onik and Rubinsky, 2010; Arena, C.B., et al., A three-dimensional in vitro tumor platform for modeling therapeutic irreversible electroporation. Biophysical Journal, 2012.103(9): p. 2033-2042 (“Arena et al., 2012”); Thomson, K.R, et al., Investigation of the Safety of Irreversible Electroporation in Humans. Journal of Vascular and Interventional Radiology, 2011. 22(5): p. 611-621; Bagla, S. and D. Papadouris, Percutaneous irreversible electroporation of surgically unresectable pancreatic cancer: a case report. Journal of Vascular and Interventional Radiology: JVIR, 2012. 23(1): p. 142-145; Ball, C., K.R. Thomson, and H. Kavnoudias, Irreversible electroporation: a new challenge in “out of-operating theater” anesthesia. Anesth Analg, 2010. 110(5): p. 13059 and Kingham, T.P., et al., Ablation of perivascular hepatic malignant tumors with irreversible electroporation. Journal of the American College of Surgeons, 2012. 215(3):p.379-387). In addition, systems and methods which utilize these electric field thresholds are needed to provide more targeted electroporation-based therapies.

### SUMMARY OF THE INVENTION

Embodiments of the invention include methods for treating a target tissue of a patient in need thereof. In one embodiment, the method may comprise one or more or all of the following steps: a) identifying a target tissue in a patient in need of treatment, b) providing or identifying one or more characteristics of one or more cells of the target tissue, c) calculating a threshold electric field for inducing IRE in the target tissue based on the one or more characteristics, which can be performed by a processor, and d) implanting one or more electrodes in the target tissue and delivering one or more electrical pulses through the electrodes based on one or more pulse parameters that produce the threshold electric field in the target tissue.

In another embodiment, the method may comprise a) identifying a target tissue in a patient in need of treatment, b) taking a biopsy of the target tissue, c) determining the size of one or more cells in a portion of the biopsy of the target tissue, d) calculating with a processor a threshold electric field for producing IRE in the target tissue based on the size of the one or more cells, and e) implanting one or more electrodes in the target tissue and delivering one or more electrical pulses through the electrodes based on one or more pulse parameters that produce the threshold electric field in the target tissue.

In embodiments, the one or more characteristics are selected from the group comprising, consisting of, or consisting essentially of: cell size, cell type, tissue type, aggressiveness of cell, shape of cell, stage of cancer, grade of cancer, and rate of cell division. The one or more cell characteristics may be any characteristic that influences the transmembrane potential of the cell during IRE. The cell size can be determined by measuring cell diameter, cell radius, cell volume, cell mass, or cell surface area. Cell size, diameter, radius, volume, mass, or surface area can be an average cell size, diameter, radius, volume, mass, or surface area for cells of a target region, or of a particular type of tissue, or patient-specific cell characteristics. The cell diameter of one or more of the tissue cells may be measured in 3D culture such as hydrogel. In some embodiments, the hydrogel comprises collagen. Additionally, the electrical field threshold for IRE may correlate with the diameter of the one or more tissue cells in the hydrogel.

In additional embodiments, the one or more characteristics of cells of the target tissue are provided in a computer memory. Alternatively or in addition, the one or more characteristics of cells in the target tissue can be identified through a biopsy of the target tissue.

In embodiments, the one or more parameters are selected from the group comprising, consisting of, or consisting essentially of: voltage, electrode spacing, electrode length, treatment duration, number of pulses, pulse width, electric field intensity, and electrode diameter.

In embodiments, the electric field threshold for IRE is calculated based on the equation:

$${E}_{\text{IRE}} = \left(1 \text{V}\right) / \left({f}_{\text{s}} R c o s \text{θ}\right)$$

wherein:
E_{IRE} is the electrical field threshold for IRE
fₛ is a shape factor reflecting the morphology and dielectric properties of the cell and surrounding media;
R is the radius of the cell.

Embodiments include a system for treatment planning for medical therapies involving administering electrical treatment energy. In embodiments the system comprises a computer comprising a memory, a processor coupled to the memory, and a treatment planning module stored in the memory and executable by the processor. The treatment planning module is adapted to receive as input one or more characteristics of cells of a target tissue, instruct the processor to calculate a threshold electric field for inducing IRE based on the one or more characteristics.

In embodiments of the systems and methods, the one or more characteristics of cells of a target tissue are selected from the group comprising, consisting of, or consisting essentially of: cell size, cell type, tissue type, aggressiveness of cell, shape of cell, stage of cancer, grade of cancer, and rate of cell division. In a specific embodiment, the one or more characteristic of cells of the target tissue are cell size which may be characterized by cell radius, cell diameter, cell volume, cell mass, or cell surface area.

In embodiments of the system, the treatment planning module is adapted to instruct the processor to calculate the threshold electric field for IRE. The treatment planning module is adapted to instruct the processor to calculate the threshold electric field for IRE according to the equation:

$${E}_{\text{IRE}} = \left(1 \text{V}\right) / \left({f}_{\text{s}} R c o s \text{θ}\right)$$

wherein:
E_{IRE} is the electrical field threshold for IRE
fₛ is a shape factor reflecting the morphology and dielectric properties of the cell and surrounding media
R is the radius of the cell

The treatment planning module can be adapted to instruct the processor to calculate one or more parameters which produce the threshold electric field for IRE. The one or more parameters are selected from the group comprising, consisting of, or consisting essentially of voltage, electrode spacing, electrode length, treatment duration, number of pulses, pulse width, electric field intensity, and electrode diameter.

Embodiments of the systems and methods of the invention customize threshold e-fields based on patient-specific cell characteristics, thereby increasing precision of the ablation zone produced by IRE and minimizing effects to surrounding healthy tissue. In addition, treatment times may be reduced. In this way, the present invention can help to minimize the inefficiencies of a one size fits all treatment protocol.

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a schematic diagram of a representative system of the invention.

FIG. 2 is a schematic diagram of a representative treatment control computer of the invention.

FIG. 3 is schematic diagram illustrating details of the generator shown in the system of FIG. 1, including elements for detecting an over-current condition.

FIG. 4A is a graph showing the applied electric field for a characteristic H-FIRE treatment.

FIG. 4B is a graph showing the applied electric field for a characteristic IRE treatment.

FIG. 4C is a graph showing the transmembrane potential (TMP) of cells of the H-FIRE treatment of FIG. 4A, while FIG. 4D is a graph showing the transmembrane potential (TMP) of cells of the IRE treatment of FIG. 4B. In the TMP plots, the dotted line represents a cell with a diameter of 15 µm and the solid line represents a cell with a diameter of 10 µm. The maximum TMP is less dependent on cell size during H-FIRE than during IRE.

FIG. 5 is a bar graph showing in vitro results of MOSE cells treated with H-FIRE and IRE in spheroidal and suspension morphologies. The threshold for cell death (taken to be the electric field that results in 5% viability) is the same for H-FIRE when applied to either morphology. However, IRE requires a higher electric field to kill cells in suspension. This is due to the smaller diameter of cells in suspension versus spheroids.

FIG. 6 is a photograph of a hydrogel with stainless steel hollow electrodes inserted. The electrodes have a center-to-center distance of 3.35 mm and have a 1.3 mm diameter.

FIG. 7A is a 3D is a schematic diagram showing reconstruction of a hydrogel setup with boundary conditions in Comsol.

FIG. 7B is a graphical representation of the electric field distribution (V/cm) for 450V applied denoting length and width measurements.

FIG. 8 is a graph showing the electric field values from the center to the edge of the hydrogel along the width dimension for various applied voltages.

FIG. 9 is a table summarizing of characteristics and results for prostate and pancreatic cancer cell lines for the experiment performed in the Example.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Reference will now be made in detail to various exemplary embodiments of the invention. Embodiments described in the description and shown in the figures are illustrative only and are not intended to limit the scope of the invention. Changes may be made in the specific embodiments described in this specification and accompanying drawings that a person of ordinary skill in the art will recognize are within the scope and spirit of the invention.

Throughout the present teachings, any and all of the features and/or components disclosed or suggested herein, explicitly or implicitly, may be practiced and/or implemented in any combination, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

Embodiments of the invention include methods for treating a target tissue of a patient in need thereof. In one embodiment, the method may comprise a) identifying a target tissue in a patient in need of treatment, b) providing or identifying one or more characteristics of one or more cells of the target tissue, c) calculating with a processor a threshold electric field for inducing IRE in the target tissue based on the one or more characteristics, d) implanting one or more electrodes in the target tissue and delivering one or more electrical pulses through the electrodes based on one or more pulse parameters that produce the threshold electric field in the target tissue.

In another embodiment, the method may comprise a) identifying a target tissue in a patient in need of treatment, b) taking a biopsy of the target tissue, c) determining the size of one or more cells in a portion of the biopsy of the target tissue d) calculating with a processor a threshold electric field for producing IRE in the target tissue based on the size of the one or more cells, and e) implanting one or more electrodes in the target tissue and delivering one or more electrical pulses through the electrodes based on one or more pulse parameters that produce the threshold electric field in the target tissue.

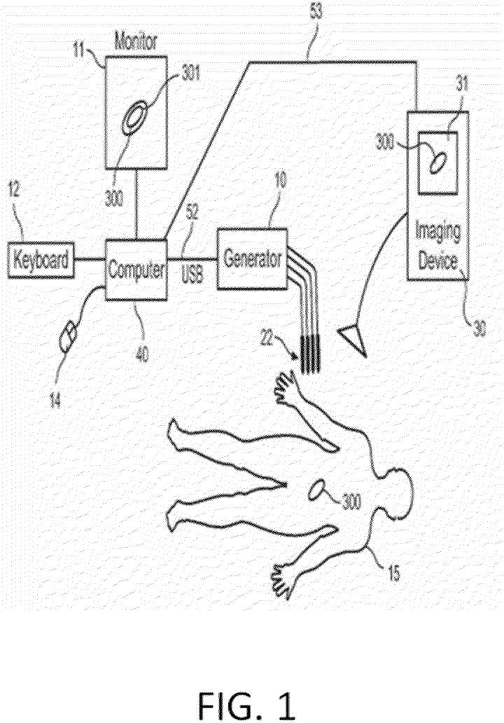

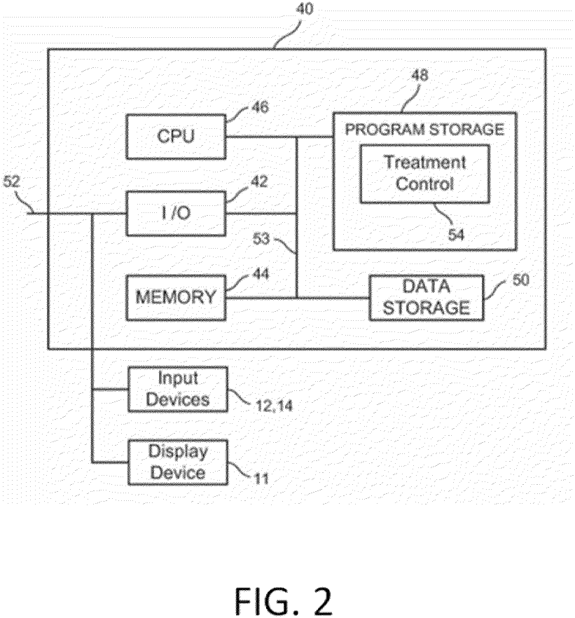

Additionally, embodiments of the invention may include one or more systems capable of performing one or more steps of the method. One embodiment of the present invention is illustrated in FIGS. 1 and 2. Representative components that can be used with the present invention can include one or more of those that are illustrated in FIG. 1. For example, in embodiments, one or more probes 22 can be used to deliver therapeutic energy and are powered by a voltage pulse generator 10 that generates high voltage pulses as therapeutic energy, such as pulses capable of irreversibly electroporating the tissue cells of the target tissue. In the embodiment shown, the voltage pulse generator 10 includes six separate receptacles for receiving up to six individual probes 22 which are adapted to be plugged into the respective receptacle. The receptacles are each labeled with a number in consecutive order. In other embodiments, the voltage pulse generator can have any number of receptacles for receiving more or less than six probes.

For example, a treatment protocol according to the invention could include a one or more or a plurality of electrodes. According to the desired treatment pattern, the plurality of electrodes can be disposed in various positions relative to one another. In a particular example, a plurality of electrodes can be disposed in a relatively circular pattern with a single electrode disposed in the interior of the circle, such as at approximately the center. Any configuration of electrodes is possible and the arrangement need not be circular but any shape periphery can be used depending on the area to be treated, including any regular or irregular polygon shape, including convex or concave polygon shapes. The single centrally located electrode can be a ground electrode while the other electrodes in the plurality can be energized. Any number of electrodes can be in the plurality such as from about 1 to 20. Indeed, even 3 electrodes can form a plurality of electrodes where one ground electrode is disposed between two electrodes capable of being energized, or 4 electrodes can be disposed in a manner to provide two electrode pairs (each pair comprising one ground and one electrode capable of being energized). During treatment, methods of treating can involve energizing the electrodes in any sequence, such as energizing one or more electrode simultaneously, and/or energizing one or more electrode in a particular sequence, such as sequentially, in an alternating pattern, in a skipping pattern, and/or energizing multiple electrodes but less than all electrodes simultaneously, for example.

In the embodiment shown, each probe 22 includes either a monopolar electrode or bipolar electrodes having two electrodes separated by an insulating sleeve. In one embodiment, if the probe includes a monopolar electrode, the amount of exposure of the active portion of the electrode can be adjusted by retracting or advancing an insulating sleeve relative to the electrode. See, for example, U.S. Pat. No. 7,344,533, which is incorporated by reference herein in its entirety. The pulse generator 10 is connected to a treatment control computer 40 having input devices such as keyboard 12 and a pointing device 14, and an output device such as a display device 11 for viewing an image of a target treatment area such as a lesion 300 surrounded by a safety margin 301. The therapeutic energy delivery device 22 is used to treat a lesion 300 inside a patient 15. An imaging device 30 includes a monitor 31 for viewing the lesion 300 inside the patient 15 in real time. Examples of imaging devices 30 include ultrasonic, CT, MRI and fluoroscopic devices as are known in the art.

The present invention includes computer software (treatment planning module 54) which assists a user to plan for, execute, and review the results of a medical treatment procedure, as will be discussed in more detail below. For example, the treatment planning module 54 assists a user to plan for a medical treatment procedure by enabling a user to more accurately position each of the probes 22 of the therapeutic energy delivery device 20 in relation to the lesion 300 in a way that will generate the most effective treatment zone. The treatment planning module 54 can display the anticipated treatment zone based on the position of the probes and the treatment parameters. Additionally, the treatment planning module 54 may have a user interface which allows a user to input one or more characteristics of cells of the target tissue and calculate a threshold electric field for IRE based on the one or more characteristics.

The treatment planning module 54 can display the progress of the treatment in real time and can display the results of the treatment procedure after it is completed. This information can be displayed in a manner such that it can be used for example by a treating physician to determine whether the treatment was successful and/or whether it is necessary or desirable to re-treat the patient.

For purposes of this application, the terms “code”, “software”, “program”, “application”, “software code”, “computer readable code”, “software module”, “module” and “software program” are used interchangeably to mean software instructions that are executable by a processor. The “user” can be any human, including for example, a physician or other medical professional. The treatment planning module 54 executed by a processor outputs various data including text and graphical data to the monitor 11 associated with the generator 10.

Referring now to FIG. 2, the treatment control computer 40 of the present invention manages planning of treatment for a patient. The computer 40 is connected to the communication link 52 through an I/O interface 42 such as a USB (universal serial bus) interface, which receives information from and sends information over the communication link 52 to the voltage generator 10. The computer 40 includes memory storage 44 such as RAM, processor (CPU) 46, program storage 48 such as ROM or EEPROM, and data storage 50 such as a hard disk, all commonly connected to each other through a bus 53. The program storage 48 stores, among others, a treatment planning module 54 which includes a user interface module that interacts with the user in planning for, executing and reviewing the result of a treatment. Any of the software program modules in the program storage 48 and data from the data storage 50 can be transferred to the memory 44 as needed and is executed by the CPU 46.

In embodiments, the user interface may be a graphical user interface which may be used in conjunction with the computer readable code. The user interface may allow a user to enter or input one or more characteristics of the cells of the target tissue to be used by the treatment planning module 54 in determining one or more parameters of a treatment protocol for IRE. Such characteristics may include cell size, cell type, tissue type, aggressiveness of cell, shape of cell, stage of cancer, and rate of cell division. The one or more cell characteristics may be any characteristic that influences the transmembrane potential of the cell during IRE. The user interface may allow such input through the use of text fields, check boxes, pull-downs, sliders, command buttons, and the like. For example, text fields allow a user to input cell diameter in terms of micrometers, or select a cell diameter based on a pull down menu that lists discrete cell diameter values. Based on this input 54, the treatment planning module 54 can calculate a threshold electric field for IRE of the target tissue and one or more parameters of a treatment protocol for administering the IRE in a manner sufficient to produce this threshold electric field.

In embodiments, the treatment planning module 54 calculates the lowest electric field for cell death based on the one or more characteristics of cells of the target tissue. As described in the Example below, the mathematical relationship between each cell characteristic and the minimum electric field for cell death can be determined experimentally. For example, the Example below arrived at the following equation for determining the minimum electric field threshold:

$${E}_{\text{IRE}} = \left(1 \text{V}\right) / \left({f}_{\text{s}} R c o s \text{θ}\right)$$

wherein:
E_{IRE} is the electrical field threshold for IRE
fₛ is a shape factor reflecting the morphology and dielectric properties of the cell and surrounding media
R is the radius of the cell

The mathematical relationships such as the equation above can be programmed into the treatment planning module 54, and can be used to calculate the electric field threshold for IRE. For example, a user may chose the radius and/or shape factor of the cells, and based on these factors, the treatment planning module 54 may calculate the electric field threshold for IRE.

In one embodiment, the computer 40 is built into the voltage generator 10. In another embodiment, the computer 40 is a separate unit which is connected to the voltage generator through the communications link 52. In a preferred embodiment, the communication link 52 is a USB link. In one embodiment, the imaging device 30 is a standalone device which is not connected to the computer 40. In the embodiment as shown in FIG. 1, the computer 40 is connected to the imaging device 30 through a communications link 53. As shown, the communication link 53 is a USB link. In this embodiment, the computer can determine the size and orientation of the lesion 300 by analyzing the data such as the image data received from the imaging device 30, and the computer 40 can display this information on the monitor 11. In this embodiment, the lesion image generated by the imaging device 30 can be directly displayed on the grid (not shown) of the display device (monitor) 11 of the computer running the treatment planning module 54. This embodiment would provide an accurate representation of the lesion image on the grid, and may eliminate the step of manually inputting the dimensions of the lesion in order to create the lesion image on the grid. This embodiment would also be useful to provide an accurate representation of the lesion image if the lesion has an irregular shape.

It should be noted that the software can be used independently of the pulse generator 10. For example, the user can plan the treatment on a different computer as will be explained below and then save the treatment parameters to an external memory device, such as a USB flash drive (not shown). Any non-transitory computer-readable media can be used to store the software and/or the output of the software for a particular treatment protocol. The data from the memory device relating to the treatment parameters can then be downloaded onto the computer 40 to be used with the generator 10 for treatment. Additionally, the software can be used for hypothetical illustration of zones of ablation, temperature thresholds or cutoffs, and electrical field thresholds or cutoffs for training purposes to the user on therapies that deliver electrical energy. For example, the data can be evaluated by a human to determine or estimate favorable treatment protocols for a particular patient rather than programmed into a device for implementing the particular protocol. The treatment protocols can be designed to produce the minimum electrical field threshold for inducing IRE calculated by the treatment planning module 54.

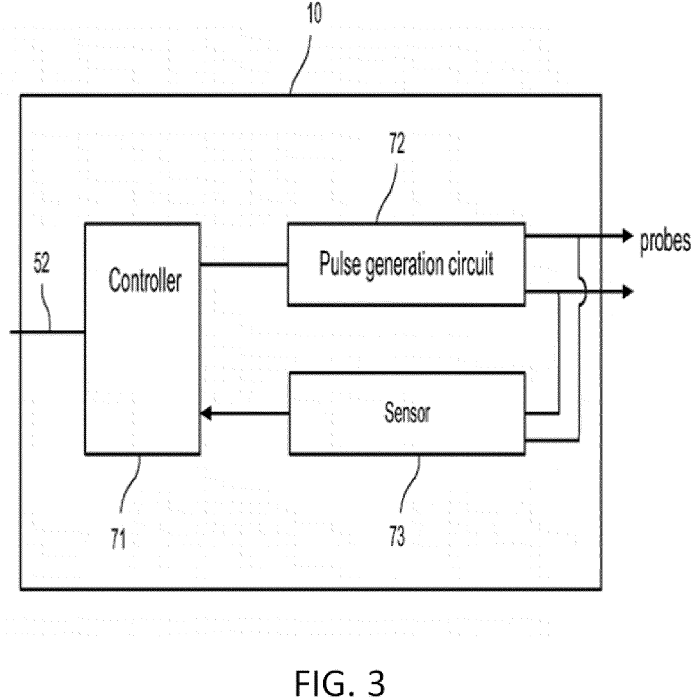

FIG. 3 illustrates one embodiment of a circuitry to detect an abnormality in the applied pulses such as a high current, low current, high voltage or low voltage condition. This circuitry is located within the generator 10 (see FIG. 1). A USB connection 52 carries instructions from the user computer 40 to a controller 71. The controller can be a computer similar to the computer 40 as shown in FIG. 2. The controller 71 can include a processor, ASIC (application-specific integrated circuit), microcontroller or wired logic. The controller 71 then sends the instructions to a pulse generation circuit 72. The pulse generation circuit 72 generates the pulses and sends electrical energy to the probes. For clarity, only one pair of probes/electrodes are shown. However, the generator 10 can accommodate any number of probes/electrodes (e.g., from 1-10, such as 6 probes) and energizing multiple electrodes simultaneously for customizing the shape of the ablation zone. In the embodiment shown, the pulses are applied one pair of electrodes at a time, and then switched to another pair. The pulse generation circuit 72 includes a switch, preferably an electronic switch that switches the probe pairs based on the instructions received from the computer 40. A sensor 73 such as a sensor can sense the current or voltage between each pair of the probes in real time and communicate such information to the controller 71, which in turn, communicates the information to the computer 40. If the sensor 73 detects an abnormal condition during treatment such as a high current or low current condition, then it will communicate with the controller 71 and the computer 40 which may cause the controller to send a signal to the pulse generation circuit 72 to discontinue the pulses for that particular pair of probes. The treatment planning module 54 can further include a feature that tracks the treatment progress and provides the user with an option to automatically retreat for low or missing pulses, or over-current pulses (see discussion below). Also, if the generator stops prematurely for any reason, the treatment planning module 54 can restart at the same point where it terminated, and administer the missing treatment pulses as part of the same treatment. In other embodiments, the treatment planning module 54 is able to detect certain errors during treatment, which include, but are not limited to, “charge failure”, “hardware failure”, “high current failure”, and “low current failure”.

General treatment protocols for the destruction (ablation) of undesirable tissue through electroporation are known. They involve the insertion (bringing) electroporation electrodes to the vicinity of the undesirable tissue and in good electrical contact with the tissue and the application of electrical pulses that cause irreversible electroporation of the cells throughout a region of or the entire area of the undesirable tissue. The cells whose membrane was irreversible permeabilized may be removed or left in situ (not removed) and as such may be gradually removed by the body’s immune system. Cell death is produced by inducing the electrical parameters of irreversible electroporation in the undesirable area.

Electroporation protocols involve the generation of electrical fields in tissue and are affected by the Joule heating of the electrical pulses. When designing tissue electroporation protocols it is important to determine the appropriate electrical parameters that will maximize tissue permeabilization without inducing deleterious thermal effects. It has been shown that substantial volumes of tissue can be electroporated with reversible electroporation without inducing damaging thermal effects to cells and these volumes have been quantified (Davalos, R. V., B. Rubinsky, and L. M. Mir, Theoretical analysis of the thermal effects during in vivo tissue electroporation. Bioelectrochemistry, 2003. Vol. 61(1-2): p. 99-107).

The electrical pulses used to induce irreversible electroporation in tissue are typically larger in magnitude and duration from the electrical pulses required for reversible electroporation. Further, the duration and strength of the pulses for irreversible electroporation are different from other methodologies using electrical pulses such as for intracellular electro-manipulation or thermal ablation. The methods are very different even when the intracellular (nano-seconds) electro-manipulation is used to cause cell death, e.g. ablate the tissue of a tumor or when the thermal effects produce damage to cells causing cell death.

Typical values for pulse length for irreversible electroporation are in a range of from about 5 microseconds to about 62,000 milliseconds or about 75 microseconds to about 20,000 milliseconds or about 100 microseconds ± 10 microseconds. This is significantly longer than the pulse length generally used in intracellular (nano-seconds) electro-manipulation which is 1 microsecond or less—see U.S. Published Pat. Application No. 2002/0010491.

The pulse is typically administered at voltage of about 100 V/cm to 7,000 V/cm or 200 V/cm to 2000 V/cm or 300 V/cm to 1000 V/cm about 600 V/cm for irreversible electroporation. This is substantially lower than that used for intracellular electro-manipulation which is about 10,000 V/cm-see U.S. Published Pat. Application No. 2002/0010491.

The voltage expressed above is the voltage gradient (voltage per centimeter). The electrodes may be different shapes and sizes and may be positioned at different distances from each other. The shape may be circular, oval, square, rectangular or irregular etc. The distance of one electrode to another may be in the range of about 0.5 to 10 cm, 1 to 5 cm, or 2-3 cm, for example. The electrode may have a surface area of 0.1-5 sq. cm or 1-2 sq. cm, for example.

The size, shape and distances of the electrodes can vary and such can change the voltage and pulse duration used. Those skilled in the art will adjust the parameters in accordance with this disclosure to obtain the desired degree of electroporation and avoid thermal damage to surrounding cells.

A primary factor in determining the effect of an electroporation procedure is the electric field to which the tissue is exposed. However, IRE protocols have a variety of electrical pulse parameters that may also affect the toxicity of the treatment. In addition to the electric field, these include pulse shape, number of pulses, pulse length, and repetition rate. The thermal effects of an IRE treatment during a pulse are a direct function of the conductivity of the tissue and the voltage to which it is exposed. Therefore, minimizing the thermal effects for a particular tissue type may be done by finding the minimum required electric field, and thus applied voltage, to kill the cells in the tissue.

To this end, pulse parameters and electrode configurations according to embodiments of the invention can include any combination of any of the following: a pulse length in the range of about 1 µs to 1 ms; a number of pulses ranging from 1 to10,000; an electric field distribution for each conductive wire pair and/or across a treatment region ranging from about 5 - 5,000 V/cm; a total electrical charge delivered by way of each conductive wire pair and/or across a treatment region of about 0.1 to about 500 mC; a frequency of pulse application ranging from about 0.001 - 100 Hz; a frequency of pulse signal ranging from about 0 - 100 MHz; a pulse shape that is square, exponential decay, sawtooth, sinusoidal, or of alternating polarity although the currently favored pulse shape is a biphasic DC pulse; a positive, negative, and neutral electrical charge pulses (changing polarity within the pulse); a resulting current in the treated tissue ranging from about 0 to about 100 amps; from 1-20 electrodes and/or electrically conductive wires; an electrode and/or electrically conductive wire separation distance ranging from about 0.1 mm to about 5 cm; and multiple sets of pulse/electrode parameters for a single treatment, including changing any of the above parameters within the same treatment, such as removing the electrodes and replacing them in different locations within the tissue or changing the number of electrodes, to specialize/customize outcome.

For example, in embodiments a pulse length in the range of about 1 µs to 1 ms, such as from about 5 µs to about 0.5 ms, or from about 10 µs to about 0.1 ms, or from about 15 µs to about 95 µs. Pulse lengths of 20 µs, 25 µs, 30 µs, 35 µs, 40 µs, 45 µs, 50 µs, 55 µs, 60 µs, 65 µs, 70 µs, 75 µs, 80 µs, 85 µs, 90 µs, 110 µs, 150 µs, or 200 µs, and so on are also acceptable. In some embodiments, the pulse duration of the electroporation-based therapy can exceed 100 µs. Any length pulse or pulse train can be administered in embodiments according to the invention. For example, pulse lengths of about 1 picosecond to 100 seconds can be used, such as from 10 picoseconds to about 10 seconds, or for example from about 100 picoseconds to about 1 second, or from 1 nanosecond to 100 milliseconds, or from about 10 nanoseconds to about 10 milliseconds, or from about 100 nanoseconds to about 1 millisecond, or from about 1 microsecond or 10 microseconds to about 100 microseconds. It is preferred in some embodiments to have a pulse length ranging from about 100 microseconds to about 1 second, such as a pulse length of about 110, or 120, or 130, or 140, or 150, or 200, or 300, or 350, or 400, or 500, or 600, or 700, or 800 or 900 microseconds, or about 1, 2, 3, 4, 5, 6, 7, 8, 9, or 10 milliseconds, or even 15, 20, 30, 40, 50, 60, 70, 80, 90, or 100 milliseconds, or even for example from about 200, 300, 400, 500, 600, 700, 800, or 900 milliseconds and so on.

The number of pulses can range for example from 5 to 5,000, or from about 10 to 2,000, or from about 20 to 1,000, or from about 30 to 500, or from about 50 to 200, or from about 75 to 150, or from about 90 to 120, or from about 95 to 110, or about 100 pulses. According to other embodiments, the number of pulses can range from about 5 to about 400 pulses, such as from about 10 to about 350 pulses, or for example from about 15 to about 300 pulses, including from about 20 to about 250 pulses, or from about 25 to about 200 pulses, such as from about 30 to about 150 pulses, for example from about 50 to about 125 pulses, such as from about 75 to about 175 pulses, or from about 90 to 110 pulses, such as about 100 pulses.

Typically, the electric field distribution for each conductive wire pair and/or across a treatment region for IRE is performed using voltages ranging for example between 1500 V/cm to 4,000 V/cm, including 1500 V/cm to 2000 V/cm, 2000 V/cm to 3000 V/cm, 3000 V/cm to 4000 V/cm, 2000 V/cm to 4000 V/cm, 2500 V/cm to 4000 V/cm, and so on. Voltages of much lower power can also be used, including using less than about 1500 V/cm. Applied fields of about 500 V/cm to 1000 V/cm can be used, or even of about 10 V/cm to about 750 V/cm, such as from about 50 V/cm to about 200 V/cm, or an electric field distribution of about 75 V/cm to about 100 V/cm. For example, in the treatment of brain tumors, typically, an applied field of less than 1000 V/cm can be used. Electrical pulse generators that can be used include those capable of delivering from 0 to about 5,000 V, such as the NanoKnife® system of AngioDynamics®, which for example can deliver from 0-3,000 V.

In another embodiment, the amplitude of the pulses of the electroporation-based therapy exceeds 2000 V/cm, including an amplitude of about 2200 V/cm, or 2500 V/cm, such as about 3000 V/cm, or 3500 V/cm, or about 4000 V/cm, such as 4500 V/cm, or about 5000 V/cm, such as about 5500 V/cm, or about 6000 V/cm, or about 6500 V/cm, such as about 7000 V/cm, or about 7500 V/cm, such as 8000 V/cm, or about 8500 V/cm, including 9000 V/cm, or about 9500 V/cm, such as about 10,000 V/cm and so on. Amplitude in the context of this specification refers to the magnitude of the electrical energy being applied using electrical pulses and which pulses can be of either positive or negative polarity.

According to methods of the invention, cycle times for pulses are set generally about 1 Hz. Furthermore, it has been found that alternating polarity of adjacent electrodes minimizes charge build up and provides a more uniform treatment zone. More specifically, in experiments performed by the inventors, a superficial focal ablative IRE lesion was created in the cranial aspect of the temporal lobe (ectosylvian gyrus) using the NanoKnife® (Angiodynamics, Queensbury, N.Y.) generator, blunt tip bipolar electrode (Angiodynamics, No. 204002XX) by delivering 9 sets of ten 50 µs pulses (voltage-to-distance ratio 2000 V/cm) with alternating polarity between the sets to prevent charge build-up on the stainless steel electrode surfaces. These parameters were determined from ex-vivo experiments on canine brain and they ensured that the charge delivered during the procedure was lower than the charge delivered to the human brain during electroconvulsive therapy (an FDA approved treatment for major depression). Excessive charge delivery to the brain can induce memory loss, and thus is preferably avoided.

Specific method embodiments may employ administering electroporation based therapy using a pulse rate of about 1 Hz to 20 GHz, such as for example from about 10 Hz to 20 GHz, or about 50 Hz to 500 Hz, or 100 Hz to 1 kHz, or 10 kHz to 100 kHz, or from 250 kHz to 10 MHz, or 500 kHz to 1 MHz, such as from 900 kHz to 2 MHz, or from about 100 MHz to about 10 GHz, including from about 200 MHz to about 15 GHz and so on.

In preferred embodiments, a total electrical charge delivered by way of each conductive wire pair and/or across a treatment region of about 0.5 to about 25 mC can be used, such as about 1 mC to about 20 mC, or from about 1.5 mC to about 15 mC, or from about 2 mC to about 10 mC, or from about 5 mC to about 8 mC, and so on. Similarly, in preferred embodiments, the resulting current in the treated tissue can range for example from about 1 A to about 8 A, or from about 2 A to about 6 A, or from about 3 A to about 5 A, such as 4 A. Indeed, for certain applications the total electrical charge delivered can range from about 0.5 to about 500 mC, such as about 10 mC to about 200 mC, or from about 15 mC to about 150 mC, or from about 20 mC to about 100 mC, or from about 50 mC to about 80 mC, and so on. The resulting current in the treated tissue can range for example from about 1 A to about 80 A, or from about 20 A to about 60 A, or from about 30 A to about 50 A, such as 40 A. It is not uncommon for currents for IRE treatments to reach or exceed 40 and 50 amps, and it is further feasible to operate under even higher current with pulse generators capable of operating under such conditions as well. Currents are expected to be high in certain applications, especially when working in an area where the tissue or the medium is highly conductive, such as with blood present in a blood vessel. Pulse width, pulse shape, number of pulses, and the resultant current in the tissue can be adjusted to achieve specific target goals for limiting the total electric charge, and any of the specific values disclosed in this specification can be used to calculate the target expected charge.

Any number of electrically conductive wires or electrodes can also be used. However, in preferred embodiments 3 to about 18 electrodes are used, such as 3 to 16, or from about 3 to 15, or from 4 to 12, or from 5 to 10, or from 6 to 8. Any one or more of the electrodes/wires can be selectively energized to achieve a particular treatment result. Further, the separation distance between electrically conductive surfaces, such as electrically conductive wires and/or electrodes, can range from about 0.2 mm to about 4 mm, such as ranging from about 0.3 mm to about 3 mm, or from about 0.4 mm to about 2 mm, or from about 0.5 mm to about 1 mm, or from about 0.8 mm to about 4 cm, such as from about 0.9 mm to about 3 cm, or from about 1.2 cm to about 2 cm, or from about 1.5 cm to about 1.8 cm, and so on.

Additional parameters of protocols that can be used in embodiments of the invention are provided in U.S. Published Pat. Application Nos. US 2007/0043345, 2009/0269317, 2011/0106221, 2012/0109122, 2013/0184702, 2013/0345697, 2014/0039489, and 2015/0088120, as well as in U.S. Pat. Nos. 8,926,606, 8,992,517, 8,814,860, 8,465484, the disclosures of each of which are hereby incorporated by reference in their entireties.

In one embodiment, the target tissue is a tumor. The tumor that may be treated according to the invention may be associated with a cancer selected from the group consisting of brain cancer, breast cancer, colon cancer, rectal cancer, endometrial cancer, cervical cancer, kidney cancer, leukemia, liver cancer, stomach cancer, esophageal cancer, oral cancer, throat cancer, tracheal cancer, lung cancer, melanoma, non-melanoma skin cancers, non-Hodgkin lymphoma, Hodgkin lymphoma, pancreatic cancer, prostate cancer, head and neck cancers, bone cancer, and thyroid cancer. However, conceivably the target tissue may include non-tumor tissue in need of removal with IRE. For example, cardiac tissue ablation for the treatment of atrial fibrillation is one non-tumor application of IRE.

One goal of clinical IRE tissue ablation treatment is to minimize damage to vital structures surrounding the target tissue. If the intensity of the electric field (e-field) used to generate IRE can be minimized to only the threshold e-field necessary to ablate the target tissue, then collateral damage to surrounding tissue can also be minimized. Further, utilizing a threshold e-filed value can help to minimize unwanted thermal effects right around the electrodes. As shown in the Example below, the present inventors have made the surprising discover that there is a correlation between cell diameter and the e-field threshold for cell death. Additionally, the present inventors have recognized that other cell characteristics such as cell type, tissue type, aggressiveness of cell, shape of cell, stage of cancer, grade of tumor, and rate of cell division may influence the e-field threshold for cell death by affecting the transmembrane potential of the cell during IRE. From one or more cell characteristics obtained from pathology samples or from parameters obtained from a database of cell characteristic information, treatment protocols can be created to customize patient specific pulse parameters that generate e-fields at the threshold for irreversibly electroporating the target tissue, thus minimizing patient expose to larger than necessary e-fields.

The e-field causes an increase in a cell’s transmembrane potential (TMP) that determines the extent of electroporation (i.e., no electroporation, reversible electroporation, or IRE). Typically, it is assumed that IRE results when the TMP reaches 1 V for a sufficient period of time (on the order of microseconds to milliseconds). The correlation between the diameter of cell in the target tissue and the TMP can be described as an analytic expression:

$$\text{TMP =} {f}_{\text{s}} E R c o s \text{θ}$$

$$E = \left(\text{TMP}\right) / \left({f}_{\text{s}} R c o s \text{θ}\right)$$

$${E}_{\text{IRE}} = \left(1 \text{V}\right) / \left({f}_{\text{s}} R c o s \text{θ}\right)$$

where fₛ is a shape factor reflecting the morphology and dielectric properties of the cell and surrounding media, E is the applied e-field, and R is the radius of the cell. When the membrane is assumed to be highly insulative compared to the surrounding media, the shape factor reduces to 3/2 for a spherical cell and 2 for a cylindrical cell oriented perpendicular to the applied field. Additionally, for non-idealized geometries, such as adherent cells with multiple attachment processes, numerical approaches can be used to determine the TMP.

Assuming a constant or known shape factor and a TMP for IRE of 1 V, the cell diameter is the only remaining unknown variable. In the past, the e-field threshold was determined after the fact by applying a specific e-field and making correlations between the size of the ablated tissue and electric field distribution within the tissue. However, this approach neglected information relating to cell morphology, which if known ahead of time, can be used to predict the e-field threshold.

In one embodiment, the treatment planning module 54 accepts user input for a cell characteristic within the target tissue, and automatically calculates the threshold e-field for inducing cell death. In another embodiment, the present invention provides a method for customizing a patient specific e-field, comprising: (a) identifying a target tissue to be treated (b) taking a biopsy of the target tissue and optionally culturing the biopsy; (c) determining one or more cell characteristics in the biopsy or in culture; (d) inputting the one or more cell characteristics into a treatment planning module operably connected to an IRE system (e) calculating the e-field threshold and corresponding pulse parameters based on the one or more cell characteristics with a processor; and (f) applying a plurality of electrical pulses to the target tissue according to the calculated pulse parameters. In embodiments, the one or more cell characteristics may include cell type, tissue type, aggressiveness of cell, shape of cell, stage of cancer, grade of tumor, and rate of cell division.

In another embodiment, the present invention provides a method for customizing a patient specific e-field, comprising: (a) identifying a target tissue to be treated (b) taking a biopsy of the target tissue and optionally culturing the biopsy; (c) measuring the size of tissue cells in the biopsy or in culture; (d) inputting the cell size into a treatment planning module operably connected to an IRE system (e) calculating the e-field threshold and corresponding pulse parameters based on the diameter with a processor; and (f) applying a plurality of electrical pulses to the target tissue according to the calculated pulse parameters. In embodiments, the cell size may be determined through measurements of cell volume, cell diameter, cell radius, cell surface area, and the like.

Biopsy procedures for different pathologies are known. Examples include bone marrow biopsies, endoscopic biopsies, and needle biopsy procedures including fine needle aspiration, core needle biopsies, vacuum-assisted biopsies, and image-guided biopsies. These would be performed according to standard protocols used when taking a biopsy for pathology, except a portion of the tissue sample would either be immediately put in culture or cryopreserved with the use of a cryoprotectant, such as DMSO or glycerol, for later culturing. Thus, the biopsy procedures need not be elaborated here. In some cases such as extensive tumors, multiple biopsies may be warranted.

The cells from the portion of the biopsy may be cultured through a variety of methods known for tissue culture, primary cell culture, and cancer cell culture. For example, for primary cell culture, the tissue sample may be first dissected to remove fatty and necrotic cells. Then, the tissue sample may be subject to enzymatic or mechanical disaggregation. The dispersed cells may then be incubated, and the media changed to remove loose debris and unattached cells. Because primary cells are anchorage-dependent, adherent cells, they require a surface in order to grow properly in vitro. In one embodiment, the cells are cultured in two-dimensional (2D) cultures. Typically, a plastic uncoated vessel such as a flask or petri dish is used, and the cells are bathed in a complete cell culture media, composed of a basal medium supplemented with appropriate growth factors and cytokines. During establishment of primary cultures, it may be useful to include an antibiotic in the growth medium to inhibit contamination introduced from the host tissue. Various protocols for culturing primary cells are known and a variety of resources are available, including the ATCC® Primary Cell Culture Guide, available on the American Type Culture Collection (ATCC) website, Human Cell Culture Protocols (Methods in Molecular Biology), Mitry, Ragai R., and Hughes, Robin D. (Eds.), 2012, and Cancer Cell Culture: Methods and Protocols (Methods in Molecular Biology) Ian A. Cree (Ed.), 2011.

In another embodiment, the cells are cultured in three-dimensional (3D) cultures. In a specific embodiment, hydrogels such as collagen-based constructs are used to culture the cells. Such hydrogels and other 3D culturing methods have been reviewed in the literature (see Tibbitt, M.W. and Anseth, K.S., Hydrogels as extracellular matrix mimics for 3D cell culture. Biotechnol Bioeng. 2009 Jul 1; 103(4): 655-663; DeVolder R., Kong H.J. Hydrogels for in vivo-like three-dimensional cellular studies. Wiley Interdiscip Rev Syst Biol Med. 2012 Jul-Aug;4(4):351-65; Gill B.J., West J.L., Modeling the tumor extracellular matrix: Tissue engineering tools repurposed towards new frontiers in cancer biology. J Biomech. 2014 Jun 27;47(9):1969-78; and Nyga A., 3D tumour models: novel in vitro approaches to cancer studies. J Cell Commun Signal. 2011 Aug; 5(3): 239-248). Collagen hydrogels comprise a random mesh of collagen fibrils supporting excess fluid. Such hydrogels are thought to more closely mimic the tumor microenvironment than 2-D constructs. Alternatively or in addition to collagen, hydrogels based on other extracellular matrix components such as hyaluronic acid, laminin, fibrin, or other biological sources such as chitosan, alginate, or silk fibrils may be used, as well as synthetic materials such as poly(ethylene glycol) (PEG), poly(vinyl alcohol) (PVA), and poly(2-hydroxy ethyl methacrylate). In one embodiment, cells from a biopsy are first cultured in tissue culture and then implanted them in a hydrogel as single cells or aggregates. Three-dimensional cell culture protocols are available, such as 3D Cell Culture- Methods and Protocols, Haycock, John (Ed.), 2011.

In one embodiment, the size of the cells in the biopsy in culture is determined. The size of the cells may be calculated as an average size, such as an average cell diameter, or a median size, such as a media cell diameter, of the sample of cells. For example, through microscopic imaging procedures, the size of the cells can be measured manually or through automated methods such as software algorithms for image cytometry known in the art. An example of such software is CellProfiler, an open-source image analysis software (see Carpenter A.E. et al.,CellProfiler: image analysis software for identifying and quantifying cell phenotypes. Genome Biol. 2006; 7(10): R100. Published online 2006 Oct 31. doi: 10.1186/gb-2006-7-10-r100).

Alternatively, the cells can be removed from culture and subject to flow cytometry analysis to measure size. Flow Cytometry one can determine relative size of cells using a known control. Typically, the FSC (Forward SCatter) parameter, which is a measurement of the amount of the laser beam that passes around the cell, can be used to give a relative size for the cell. If one uses a known control or standard such as beads with a known size, one can determine the relative size of the cells based on the size of the control or standard.

Alternatively, or in addition, COULTER COUNTERS®, which measure changes in impedance between electrodes, can be used to measure number, cell volume, mass and surface area size distributions. If the morphology of the cells is known to be approximately round, cell diameter may be inferred from these measurements. Further, CASY® cell counting technology can provide a highly accurate measurement of cell volume.

In other embodiments, a portion of the biopsy is subject to standard pathology protocols. For example, if the tissue to be treated is a tumor, a biopsy can be conducted to determine such characteristics as the stage of the tumor and other pathology features including the type of cancer cells, the grade of tumor (how abnormal the tumor cells and the tumor tissue look under a microscope), the presence of tumor markers, and cytogenetic features. One or more of these characteristics may then be entered into the treatment planning module 54, and a custom e-field threshold for the target tissue to be treated with IRE may be determined. Additional features that may be determined from a biopsy include tissue type (i.e. epithelial, connective, nervous, and muscle) and cell type (e.g. squamous, cuboidal, columnar).

Embodiments of treatment methods for customizing threshold e-fields based on patient-specific characteristics such as cell size and other cell characteristics have several advantages. For example, by identifying a threshold e-field for ablation of target tissue, damage to surrounding healthy tissue and vital structures is minimized. Further, unwanted thermal effects near the electrodes are also minimized since a minimum energy level is being applied through the electrodes. In addition, embodiments of the present invention allow treatment times to be reduced, depending on the pulse parameters that will be required to generate the threshold e-field. Since the e-field may be based on a patient-specific parameter such as cell size, instead of a more generic category such as tissue type, pathologic variability from patient to patient can be accounted for in creating a customized treatment. In other words, the methods of the present invention can minimize inefficiencies of a one size fits all treatment protocol.

However, other embodiments may rely on population-specific information for the cell characteristics described herein. For example, cell size and other cell characteristics of specific tissue or tumor types may be collected and stored in a database in computer memory 44. This information may be obtained from biopsy samples archived at hospitals or pathology laboratories or from previous studies in the scientific literature, or from tumor registries that collect this information. In this way, once the tissue type or tumor type for electroporation is known, these can be inputted into the treatment planning module 54 and one or more cell characteristics such as cell size may be retrieved from the database. The cell size and other cell characteristics appropriate for the target tissue can them be used in calculating the e-field threshold and corresponding pulse parameters. However, this method has the disadvantage over the previous method in that it generalizes to a particular tissue type rather than providing a patient-specific parameter.

In an additional embodiment, the cultured cells may be treated with IRE in a 3D hydrogel. By treating cultured cells in 3D hydrogel, one can accurately predict how the final lesion grows over a period of time. So although lesion sizes can be tracked in real-time during a procedure in a patient, it may otherwise be difficult to predict how large a lesion will grow between 2 and 48 hours following the initial procedure. However, since the hydrogel model in the Example below reflects a correlation between cell diameter and the final lesion size (after it has finished growing), medical professionals can more accurately determine pulse parameters based a desired final lesion size. The Example below showed that IRE treatment of cultured cells in the 3D hydrogel produced five zones: cell lysis near the electrodes, IRE just beyond, gradual IRE just beyond that zone (reversible but cells cannot recover from loss of homeostasis), reversible and recoverable, and no effect. Such knowledge of how the lesion develops can be used strategically to increase the ablation volume beyond current IRE strategies.

For example, one embodiment of the invention comprises a method of predicting the final lesion size of a target tissue as a result of applying IRE, comprising: a) identifying a target tissue in a patient in need of treatment, b) taking a biopsy of the target tissue, c) determining the size of one or more cells in a portion of the biopsy of the target tissue d) calculating with a processor a final lesion size in the target tissue based on the size of the one or more cells. In embodiments, the method may further comprise e) implanting one or more electrodes in the target tissue and delivering one or more electrical pulses through the electrodes based on one or more pulse parameters that produce the final lesion size in the target tissue.

For example, another embodiment of the invention comprises a method of predicting the final lesion size of a target tissue as a result of applying IRE, comprising: a) identifying a target tissue of a patient in need of treatment b) taking a biopsy of the target tissue c) culturing a portion of the biopsy in a 3D hydrogel d) applying an electric field in the hydrogel of sufficient intensity to induce cell death of the cultured biopsy and e) measuring a final lesion size in the 3D hydrogel induced by IRE. Additionally, the method may comprise f) developing a treatment protocol for IRE comprising one or more pulse parameters based on the final lesion size in the hydrogel.

In an additional embodiment, patient specific treatment protocols can be customized based on the proposition that cells which divide more rapidly are more susceptible to IRE. As already known, cells subjected to an e-field will start to weaken. It is believed, however, that dividing cells in particular have a lower IRE susceptibility threshold than regular cells. The theory is that cells already weakened by an e-field which then try to divide become so further weakened that they cannot survive and die. Thus, a treatment protocol can take into account whether or not the target tissue is composed of dividing cells, such as cancer cells, and also how aggressively the cells are dividing. A more rapidly dividing, aggressive tumor would therefore be more susceptible to IRE than the surrounding healthy cells that are not dividing. Such information can be inputted into the treatment planning module 54 of the invention.

In an additional embodiment, the shape or morphology of the cells in the target tissue zone is used to design the treatment planning protocol. Some cells are more circular in shape, while others, such as nerve cells or smooth muscle tissue are elongated. If the shape factor is unknown, one could reconstruct the geometry of individual cells within the biopsy or culture and use numerical techniques (e.g., finite elements method or finite differences) to predict the e-field threshold. Other variables that affect the accurate determination of the shape factor include the dielectric and mechanical properties of the cell membrane. Embodiments of the invention may use dielectric spectroscopy and atomic force microscopy on cells isolated from biopsies to obtain this information. For example, metastatic cells capable of extravasation from the primary tumor are less rigid and are associated with a change in membrane conductivity and specific capacitance. This makes them more susceptible to IRE. The full definition for the shape factor is:

$${f}_{\text{s}} = \frac{3 {\text{λ}}_{o} \left[3 d {R}^{2} {\text{λ}}_{i} + \left(3 {d}^{2} R - {d}^{3}\right) \left({\text{λ}}_{\text{m}} - {\text{λ}}_{i}\right)\right]}{2 {R}^{3} \left({\text{λ}}_{\text{m}} + 2 {\text{λ}}_{o}\right) \left({\text{λ}}_{\text{m}} + \frac{1}{2} {\text{λ}}_{i}\right) - 2 {\left(R - d\right)}^{3} \left({\text{λ}}_{o} - {\text{λ}}_{\text{m}}\right) \left({\text{λ}}_{i} - {\text{λ}}_{\text{m}}\right)}$$

where λ is the conductivity of the extracellular space (o), intracellular space (i), and membrane (m). Embodiments of the method for treating a target tissue of patient following a biopsy of the target tissue can include consideration of one or moreof the factors mentioned above, including cell size, tissue type, cell type, aggressiveness of cell, shape of cell, stage of cancer, grade of tumor, and rate of cell division. One or more of these factors can be entered into the treatment planning module 54 in communication with the IRE system and generator, and a threshold electric field and customized pulse parameters corresponding to the threshold electric field can be calculated. For further discussion on correlations between increases in specific capacitance and the aggressiveness of cells, see Salmanzadeh et al., Biomicrofiuidics 7, 011809 (2013).

Further, the present inventors have obtained experimental and theoretical evidence that individual pulses with durations one to two orders of magnitude shorter than IRE pulses can kill cells in such a way that is less dependent on the cell diameter. The individual pulses are applied in alternating polarity to reduce muscle contractions. Additionally, the individual pulses are repeated to form a high-frequency burst, and multiple bursts are necessary to induce cell death. This is similar to how multiple, longer duration pulses are applied during an IRE treatment. This form of high frequency irreversible electroporation (H-FIRE) requires a higher e-field threshold, but there is less dependence on cell size. Therefore, treatment planning is significantly reduced, as different cell types, regardless of their morphology, have the same e-field threshold.

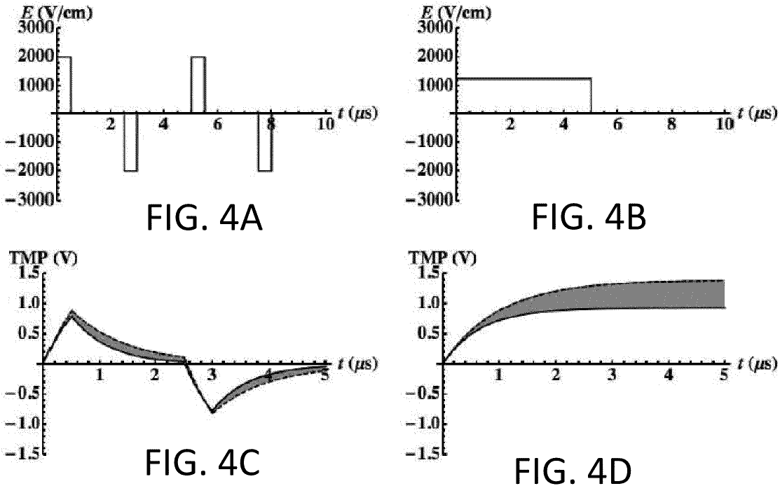

In a theoretical example, the cells are assumed to be spherical, and the conductivities λₒ and λ_{¡} are set to 0.1 S/m and λₘ is set to 3e-7 S/m. The plots in FIGS. 4C and 4D show the TMP at the cell pole (θ = 0) for a cell with a diameter of 10 µm (solid line) and a cell with a diameter of 15 µm (dotted line). On the left, the cells are exposed to H-FIRE with 500 ns long pulses applied at 2000 V/cm as shown in FIG. 4A. On the right, the cells are exposed to IRE with 5 µs long pulses applied at 1250 V/cm ad shown in FIG. 4B.

Because H-FIRE pulses are shorter than the membrane charging time, the peak TMP reached at the end of the pulses is nearly the same for both cell diameters. During IRE, the membrane is fully charged and the TMP reaches a plateau significantly different for each cell diameter (with the larger cell being easier to electroporate).

This concept has also been proved experimentally. Mouse ovarian surface epithelial (MOSE) cells where treated with H-FIRE and IRE in suspension using a high-throughput electroporation well plate. In one group, the MOSE cells were grown as tumor spheroid, which permitted the cells to self-adhere and extend to larger diameters with complex morphologies. In a second group, the tumor spheroids were digested using trypsin-EDTA and treated as individual spherical cells with a smaller diameter. Viability was assessed using an Alamar Blue metabolic assay.

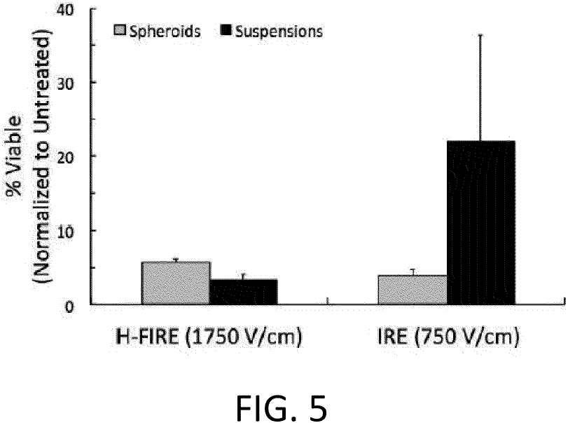

The results are shown in FIG. 5. For interpretation of the results, it is assumed that treatments resulting in less than 10% viability correspond to complete cell death. During IRE at 750 V/cm, spheroids containing large cells were killed, where as individual cell suspensions containing smaller cells were not. Alternatively, H-FIRE produced complete cell death irrespective of the cell morphology, albeit at a larger applied e-field (1750 V/cm). These results suggest that treatment planning procedures for H-FIRE do not need to include protocols for obtaining patient biopsies and measuring cell diameters.

For more background and examples of pulse parameters that can be used with H-FIRE, see U.S. Published Pat. Application No. US 2010/0261994.

Additional embodiments of the invention are listed below:

Embodiment A. A method for killing tissue cells comprising (a) positioning an electrode into or adjacent to a target zone of tissue cells; and (b) applying a plurality of electrical pulses through the electrode; wherein each of the plurality of electrical pulses is under the transmembrane potential threshold for electroporating tissue cells in the target zone, and wherein the plurality of electrical pulses is sufficient for electroporating the tissue cells in the target zone.

Embodiment B. The method of Embodiment A or any embodiment disclosed in this specification, wherein the plurality of electrical pulses are between 10,000 nanoseconds and 1 picosecond.

Embodiment C. The method of any of Embodiment A, or B, or any embodiment disclosed in this specification, wherein two or more electrodes are used in the method and are provided as part of a single device.

Embodiment D. The method of any of Embodiment A, B, C, or any embodiment disclosed in this specification, wherein two or more electrodes are used in the method.

Embodiment E. The method of any of Embodiment A-D or any embodiment disclosed in this specification, further comprising positioning the electrodes at a distance apart from each other to create custom treatment area shapes through varying electrode activation patterns.

Embodiment F. The method of any of Embodiment A-E or any embodiment disclosed in this specification, wherein applying the plurality of electrical pulses comprises emitting multiple electric pulses such that the temporal and spatial summation of such pulses results in the generation of an electric field of about 500 V/cm to 2500 V/cm for 10000 microseconds or less to induce IRE.

Embodiment G. The method of any of Embodiment A-F or any embodiment disclosed in this specification, wherein applying multiple electrical pulses comprises emitting multiple electric pulses such that the temporal and spatial summation of such pulses results in the generation of an electric field of about 1 kV/cm to 50 kV/cm for 1000 nanoseconds or less to induce supra-poration in addition to IRE.

Embodiment H. The method of any of Embodiment A-G or any embodiment disclosed in this specification, wherein individual electric pulses of the plurality of electrical pulses are monophasic.

Embodiment I. The method of any of Embodiment A-H, wherein individual electric pulses of the plurality of electrical pulses are biphasic.

Embodiment J. The method of any of Embodiment A-I, wherein a train of monophasic pulses is delivered in one direction, followed by a subsequent pulse train of opposite polarity.

Embodiment K. The method of any of Embodiment A-J, wherein waveforms of the plurality of electric pulses are triangular, square, sinusoidal, exponential, or trapezoidal.

### EXAMPLE

The following Example uses an in vitro tumor model to characterize cell death for various tissues. This model offers a more in vivo like environment for obtaining cell thresholds over traditional in vitro studies without the immediate need for animal models. Additionally, the model investigates the lesion growth versus time as different cell lines require varying amounts of time to reach a fully developed lesion as well as growth kinetics for the cell lines chosen.

### Methods

Collagen I extraction and formation of in vitro tumors were followed as previously stated (Arena et al., 2012; and Szot, C.S., et al., 3D in vitro bioengineered tumors based on collagen I hydrogels. Biomaterials, 2011. 32(31): p. 7905-7912). Rat tail tendons from Sprague Dawley rats were extracted and dissolved overnight in pH 2.0, 0.1 M hydrochloric acid with agitation. The collagen I was centrifuged at 30,000 × g, 4° C. for 45 minutes, the supernatant decanted and set volumes removed for concentration determination. Weighed volumes were dried at 75° C. overnight and re-weighed once dry. The collagen was sterilized by layering over chloroform overnight, and the final collagen batch was stored at 4° C. until the time of use. A neutralizing buffer consisting of 10 X DMEM, 1 N NaOH, and dH₂0 was used to re-suspend a cell pellet to achieve a final cell seeding density of 5 × 10⁶ cells/ml. Cells were grown and passaged according to ATCC cell protocols. The neutralizing buffer/cell solution remained on ice prior to mixing with collagen to achieve a final collagen concentration of 8 mg/ml. Six well plates were used to pipette 235 µL into 10 mm diameter cylindrical PDMS molds. After polymerization for 20 minutes at 37° C., molds were removed from the hydrogels and 2 mL of media added to each well to maintain cell viability. Hydrogels were incubated overnight in media before IRE treatment the following day.

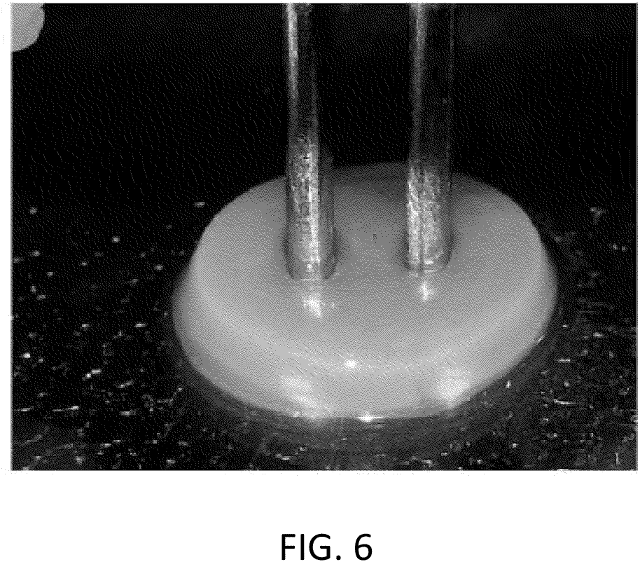

Following incubation, IRE treatment was performed using 1.3 mm diameter stainless steel hollow needles with a 3.35 mm center-to-center spacing as seen in FIG. 6. Media was aspirated from wells and IRE pulses were delivered for 100 µs long pulses for a total of 80 pulses with a repetition rate of 1 pulse/second using an ECM 830 electroporation system (BTX-Harvard Apparatus, Holliston, MA). Pulses were applied for voltages of 300 V, 375 V, and 450 V and pulse parameters were chosen based on existing IRE protocols (Arena et al., 2012; Onik, G., P. Mikus, and B. Rubinsky, Irreversible electroporation: implications for prostate ablation. Technol Cancer Res Treat, 2007. 6(4): p. 295-300; and Al-Sakere, B., et al., Tumor ablation with irreversible electroporation. PLoS ONE, 2007 2(11): p. e1135). Needle electrodes were inserted into the center of hydrogels for pulse delivery and cleaned with ethanol in between each hydrogel. Electrodes were inserted into control hydrogels without pulsing in order to validate that any observed effects resulted from irreversible electroporation only. Post-treatment, media was added to the well plates and hydrogels were incubated from a range of 2 hours to 96 hours to eliminate effects due to reversible electroporation before post-IRE analysis.

Post-IRE pulse delivery and incubation time, cell viability was assessed using a calcein acetomethoxy (AM)/propidium iodide (PI) live/dead assay. Calcein AM (4 µM; λₑₘ=515 nm; Invitrogen) was added at a concentration of 2 µl/ml to media 25 minutes prior to analysis, and propidium iodide (1.5 mM; λₑₘ = 617 nm; Invitrogen) was added at a concentration of 30 µl/ml to media five minutes before analysis. Calcein AM stains live cells as it can be transported across the cell membrane, fluorescing green once intracellular esterases remove the AM group. Propidium iodide stains dead cells as it binds to nucleic acids emitting a red fluorescence. Staining was staggered five minutes between wells to insure that dye was added for the same amount of time to each sample prior to imaging.

Hydrogels were imaged using a Leica DMI 6000 fluorescent microscope (Leica Microsystems, Buffalo Grove, IL), tiling a set of images in order to reconstruct the entire hydrogel surface. Hydrogels were removed from the six well plate using forceps and placed upside down on a petri dish for imaging. A GFP filter was used to visualize the live cells while TXR was used to visualize the dead cells. Exposure, intensity, and gain were set for each filter to minimize saturation. Four points along the edges of the hydrogel were chosen in the tiling program in order to reconstruct the hydrogel surface. Live images and dead images were overlaid on top of each other to show the lesion volume, and measurements of the lesion dimensions were taken in the LAS software.

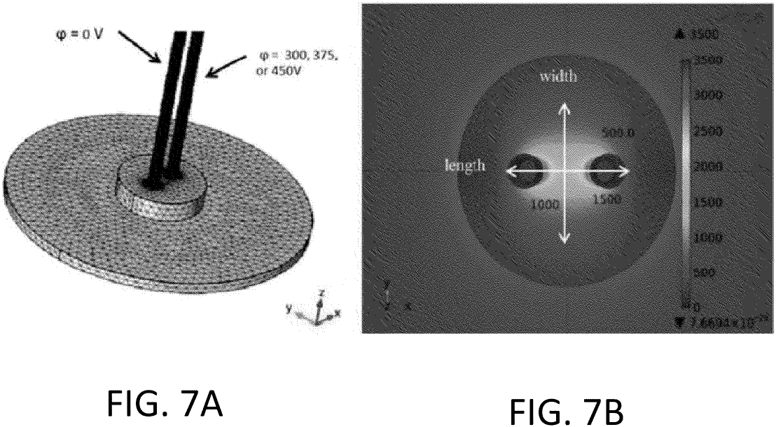

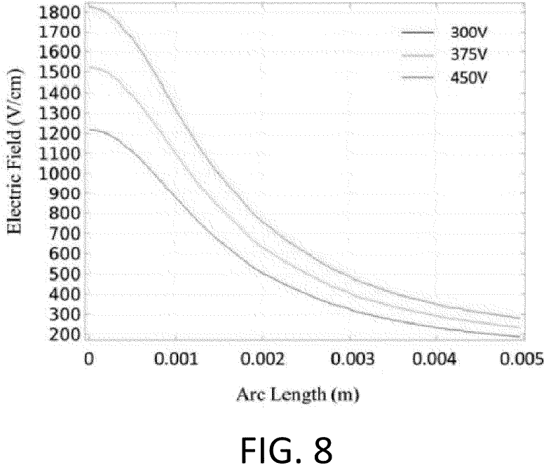

Calculations of the electric field threshold required for cell death were determined using finite element analysis (COMSOL Multiphysics 4.2a, Stockholm, Sweden). The model reconstructed the 3-D geometry of the hydrogel and electrode setup (FIG. 7A), and the electric field (FIG. 7B) was calculated at points from the center to the edge along the hydrogel. The hydrogel consists of a large cylinder representing the well plate with a radius of 1.7 cm with the hydrogel sitting in the center at x=y=z=0. The hydrogel is 10 mm in diameter and 2.25 mm in height. Two needles electrodes of infinite length and a diameter of 1.3 mm are inserted in the center of the hydrogel with a center-to-center spacing of 3.35 mm. Material properties are applied to all components by assigning electrical conductivity and relative permittivity values for the hydrogel, well plate, and needle electrodes. The well plate has an electrical conductivity of 1e-16 S/m, while the electrodes are made of stainless steel and have an electrical conductivity of 2.22e-6 S/m, and the hydrogel has an electrical conductivity of 1.2 S/m. Boundary conditions were set to specify electric potentials in which one electrode is electrically insulating with Ψ = 0 V, while the other is electrically conductive with Ψ = 0 V. The remainder of the boundaries in the model, including the hydrogel and well plate are set as insulation. An extremely fine user controlled mesh was used resulting in a <0.01% difference in electric field calculations with successive refinements. The electric field distribution is numerically solved from Laplace’s Equation:

$${∇}^{2} \text{φ} \left(\text{z}\right) = 0$$

where Ψ is the electric potential. A parametric study on voltage was performed to determine the electric fields along points across the hydrogel for 300 V, 375 V, and 450 V. A line graph was drawn from the center of the hydrogel at x=y=0 and z= (height of the hydrogel/2) which is directly in the center of the electrodes, and therefore, in the center of the electric field distribution (FIG. 8).

Electric field values for the applied voltages were solved at points along the line. Experimental width measurements of the lesion area were then correlated with the numerical model to determine the electric field thresholds for cell death at the transition between live and dead cells as previously described (Arena et al., 2012).

### Results

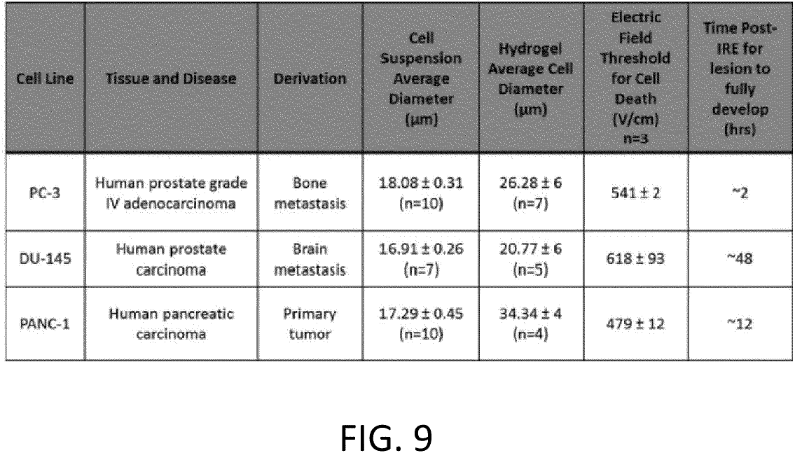

Two human pancreatic cancer cell lines (PANC-I and BxPC-3) and two human prostate cancer cell lines (PC-3 and DU-145) were used in these experiments to investigate electric field distribution and lesion growth with time using irreversible electroporation. The thresholds necessary to kill pancreatic and prostate cancer cells have not been previously investigated. The hydrogel model is a cheaper, preliminary model to investigate the thresholds for cell death with benefits over cell suspension studies as cells behave in a more in vivo like manner. The hydrogel model was successfully used to determine the irreversible electroporation thresholds for cell death once lesions had become fully developed for prostate and pancreatic cancer cell lines. As shown in the table in FIG. 9, the electric field threshold for cell death varied between cell lines as did the time for the lesions to fully develop in the hydrogel post-IRE pulse delivery. Larger cells required lower electric field thresholds for cell death. The PANC-I cells had the largest diameter in the hydrogel and as anticipated, required the lowest electric field threshold for cell death. PC-3 and DU-145 cells had higher electric field thresholds for cell death as they were smaller in size than the PANC-I cells with BxPC-3 cells requiring the highest threshold to achieve cell death.

Additionally, it was found that lesion size grew over time and differed between cell lines as can be seen in the table in FIG. 9. Lesion size continued to grow over a range of 2 to 48 hours depending on the cell line being ablated, with PC-3 cells developing a stable lesion 2 hours post-IRE and DU-145 cells 48 hours post-IRE. The width of the lesions, as depicted in FIG. 7B, grew with time while the length dimension remained relatively constant.

### Conclusions

This work further developed the use of an in vitro tumor construct to study the effects of IRE on cancer cells with a focus on the electric field threshold for cell death and the time for lesions to stop growing, reaching a fully developed state. The in vitro tumor construct is a more physiological model than cell suspension studies negating the immediate need for in vivo animal or tissue models. Additionally, the electric field threshold for various cancer cell lines can be determined through this model without the added challenge of estimating the change in conductivity that is necessary for animal and tissue models.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1-19. (canceled)

20. A method for treating a target tissue, the method comprising:
performing a biopsy of a region of interest of a subject and identifying from the biopsy that the region of interest comprises patient cells of a specific tissue type or specific tumor type;
administering a plurality of electrical pulses to the region of interest and irreversibly electroporating one or more of the patient cells of the specific tissue type or specific tumor type while not irreversibly electroporating cells of one or more other type in the region of interest.

21. The method of claim 20, wherein the patient cells of the specific tissue type or specific tumor type are cancer cells and the one or more other type of cells are non-cancerous.

22. The method of claim 21, wherein the cancer cells are pancreatic, prostate, brain or breast cancer cells.

23. The method of claim 21, further comprising:
identifying from the biopsy one or more of aggressiveness, rate of cell division, or stage of cancer of the cancer cells;
administering the plurality of electrical pulses using a combination of a specific pulse width range, voltage range, electrode spacing range, frequency range, range of number of pulses, and range of electrode diameters that will produce an electric field:
(i) that is at or above a minimum electric field required to kill cells having the identified aggressiveness, rate of cell division, or stage of cancer;
(ii) but that has a magnitude lower than required to kill the other type of cells which are less aggressive, a slower rate of or no cell division, or a lower stage of cancer.

24. The method of claim 20, further comprising:
performing one or more measurements of the patient cells of the specific tissue type or specific tumor type and constructing from the measurement(s) a morphology of the patient cells;
administering the plurality of electrical pulses to the region of interest and irreversibly electroporating only cells with the constructed cell morphology present in the region of interest and not killing the cells of the other type, which have a different cell morphology.

25. The method of claim 24, wherein cell morphology is based on one or more of cell diameter, cell radius, cell shape, cell volume, cell mass, or cell surface area.

26. The method of claim 24, wherein the administering comprises using a combination of a specific pulse width range, voltage range, electrode spacing range, frequency range, range of number of pulses, and range of electrode diameters to achieve an applied electric field that will kill one or more of the cells having the constructed cell morphology but that is of a magnitude lower than needed to kill the other type of cells with the different cell morphology.

27. The method of claim 24, further comprising:
measuring size of the patient cells of the specific tissue type or specific tumor type; or
estimating the size based on the size of previously measured cells of the same specific tissue type or the specific tumor type; and
administering the plurality of electrical pulses at or above the minimum electric field required to cause IRE of cells having the measured or estimated cell size but below the minimum electric field required to cause IRE of the other cells.

28. The method of claim 27, wherein the cell size is the cell diameter or cell radius.

29. The method of claim 27, wherein the minimum electric field is determined by:
$${E}_{\text{IRE}} = \left(\text{1 V}\right) / \left({f}_{\text{s}} R c o s \text{θ}\right)$$
wherein:
E_{IRE} is the electrical field threshold for IRE;
fₛ is a shape factor reflecting the morphology and dielectric properties the cells and surrounding media; and
R is the radius of the cells.

30. The method of claim 27, wherein the size of the patient cells of the specific tissue type or specific tumor type is measured and expressed as an average or median size, or the size of the patient cells of the specific tissue type or specific tumor type is an average or median cell size of previously measured cells of the same specific tissue type or the specific tumor type.

31. A method for treating a target tissue, the method comprising:
identifying a target region of a subject;
performing a biopsy of a portion of the target region;
identifying from the biopsy a specific cell type present in the target region;
administering a plurality of electrical pulses to the target region with a pulse protocol sufficient to cause irreversible electroporation (IRE) of cells of the specific cell type and insufficient to cause IRE of a second cell type in the target region.

32. The method of claim 31, wherein the administering comprises administering the plurality of electrical pulses in a manner to provide a voltage gradient in the target region that is lower than the minimum voltage gradient for causing IRE of the second type of cells.

33. The method of claim 31, wherein the pulse protocol for the IRE comprises administering the plurality of electrical pulses using a voltage of 5 to 450 V.

34. A method for treating a target tissue of a patient with irreversible electroporation (IRE), the method comprising:
identifying through a biopsy morphology of selected cells present within a spatial region of interest of a patient;
selecting a magnitude of an applied electric field within the range of:
a first magnitude equal to or above a magnitude of a first minimum electric field sufficient to kill by way of irreversible electroporation cells having the morphology of the selected cells; and
up to but below a second magnitude of a second minimum electric field sufficient to kill by way of irreversible electroporation other cells that are present within the spatial region of interest and that have a morphology that is different from the morphology of the selected cells;
administering a plurality of electrical pulses to the spatial region of interest using selected pulse width, voltage, electrode spacing, frequency, number of pulses, and electrode diameter, together capable of producing the applied electric field at the selected magnitude;
thereby killing with irreversible electroporation cells having the morphology of the selected cells, while not killing with irreversible electroporation the other cells.

35. The method of claim 34, wherein the cells having the morphology of the selected cells are pancreatic, prostate, brain or breast cancer cells and the other cells are non-cancerous.

36. The method of claim 34, wherein:
the pulse width is in the range of from 5 microseconds to 62,000 milliseconds; and
each pulse of the plurality of electrical pulses produces an electric field below the transmembrane potential threshold for electroporating the cells having the morphology of the selected cells.

37. The method of claim 34, where the selected voltage is 5 V to 450 V.

38. The method of claim 34, wherein cell morphology is based on one or more of cell diameter, cell radius, cell shape, cell volume, cell mass, or cell surface area.

39. The method of claim 34, wherein the selected cells are cancer cells.

40. The method of claim 34, wherein the selected cells are dividing or are dividing more rapidly than the other cells that are present within the spatial region of interest.

41. The method of claim 34, wherein the plurality of electrical pulses is administered such that the temporal and spatial summation of such pulses results in generation of an electric field of about 500 V/cm to 2500 V/cm for 10000 microseconds or less.

42. The method of claim 34, wherein the plurality of electrical pulses comprises monophasic electrical pulses.

43. The method of claim 34, wherein the plurality of electrical pulses comprises biphasic electrical pulses.

44. The method of claim 34, wherein the magnitude of the applied electric field is an amount up to 750 V/cm.

45. The method of claim 34, wherein:
the selected voltage is 300 V to 450 V; and
the magnitude of the applied electric field is an amount up to 750 V/cm.
